(19) United States
(12) Patent Application Publication
(10) Pub. No.: US 2021/0346671 A1
(43) Pub. Date: Nov. 11, 2021

(54) Tubing Connection Adaptor System

(71) Applicant: Robert Dillon, Sedro Woolley, WA (US)

(72) Inventor: Robert Dillon, Sedro Woolley, WA (US)

(21) Appl. No.: 17/314,129

(22) Filed: May 7, 2021

Related U.S. Application Data
(60) Provisional application No. 63/021,736, filed on May 8, 2020.

(51) Int. Cl. A61M 39/10

(52) U.S. Cl. CPC A61M 39/1011; A61M 2039/1033; A61M 2039/1077

(57) ABSTRACT

A tubing connection adaptor system is provided. The system includes a male connector body having a first end opposite a second end and a female connector body having a front end opposite a rear end. The front end of the female connector body is dimensioned to operably engage the first end of the male connector body therein, such that a continuous channel is defined through the male connector body and the female connector body upon engagement therebetween. A tab extends from the first end of the male connector body, wherein the tab frictionally engages a notch disposed through the front end of the female connector body.

### CROSS REFERENCE TO RELATED APPLICATIONS

This application claims the benefit of U.S. Provisional Application No. 63/021,736 filed on May 8, 2020. The above identified patent application is herein incorporated by reference in its entirety to provide continuity of disclosure.

### BACKGROUND OF THE INVENTION

The present invention relates to tubing connection systems. More particularly, the present invention pertains to a tubing connection adaptor system wherein a tap frictionally engages a notch for quick and efficient connection and disconnection of various tubing systems.

Many individuals require catheterization due to various medical conditions, however the process of connecting catheter tubes to a urine collection bag can be a difficult process. Typically, throughout the course of a standard day, the individual must frequently disconnect and reconnect the catheter tubing, such as when replacing urine collection bags. However, standard connection mechanisms require excess force to be applied to the tubing between the catheter and the urine collection bag, which can stretch and weaken the tubing. If the tubing becomes sufficiently weak, leaks can result, which can be difficult and uncomfortable to clean. Additionally, if the catheter itself is weakened, the catheter may need to be replaced, which can be a painful or uncomfortable process in itself. Therefore, an adaptor that allows a user to connect and disconnect medical tubing quickly and efficiently when necessary is desired.

In light of the devices disclosed in the known art, it is submitted that the present invention substantially diverges in design elements from the known art and consequently it is clear that there is a need in the art for an improvement to existing tubing connection systems. In this regard, the instant invention substantially fulfills these needs.

### SUMMARY OF THE INVENTION

In view of the foregoing disadvantages inherent in the known types of tubing connection systems now present in the known art, the present invention provides a tubing connection adaptor system wherein the same can be utilized for providing convenience for the user when rapidly connecting and disconnecting various tubing systems, such as those between a catheter and urine collection bag.

The present system comprises a male connector body having a first end opposite a second end and a female connector body having a front end opposite a rear end. The front end of the female connector body is dimensioned to operably engage the first end of the male connector body therein. A continuous channel is defined through the male connector body and the female connector body upon engagement therebetween. A tab extends from the first end of the male connector body, wherein the tab frictionally engages a notch disposed through the front end of the female connector body.

In some embodiments, the first end comprises a greater diameter than the second end. In another embodiment, the second end defines a tubular member extending into an interior of the second end. In other embodiments, the tab extends from a distal end of the tubular member through the first end. In yet another embodiment, a cutout extends from a forward edge of the first end and terminates at the tab. In some embodiments, a plurality of ridges is disposed about an exterior surface of each of the first end and the front end. In another embodiment, the plurality of ridges is concentrically disposed along each of the first end and the front end. In other embodiments, each ridge of the plurality of ridges defines an arcuate exterior. In yet another embodiment, the notch comprises a first leg and a second leg, wherein the first leg is disposed perpendicularly from the second leg. In some embodiments, the second end and the rear end comprise an identical diameter. In another embodiment, the front end comprises a diameter greater than the rear end. In other embodiments, a third leg extends perpendicularly from a terminal end of the second leg.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1A shows a perspective view of the male connector body of an embodiment of the tubing connection adaptor system.

FIG. 1B shows a perspective view of the female connector body of an embodiment of the tubing connection adaptor system.

FIG. 2 shows a perspective view of an embodiment of the tubing connection adaptor system in a partially connected configuration.

FIG. 3 shows an exploded view of an embodiment of the tubing connection adaptor system in use.

### DETAILED DESCRIPTION OF THE INVENTION

Reference is made herein to the attached drawings. Like reference numerals are used throughout the drawings to depict like or similar elements of the tubing connection adaptor system. The figures are intended for representative purposes only and should not be considered to be limiting in any respect.

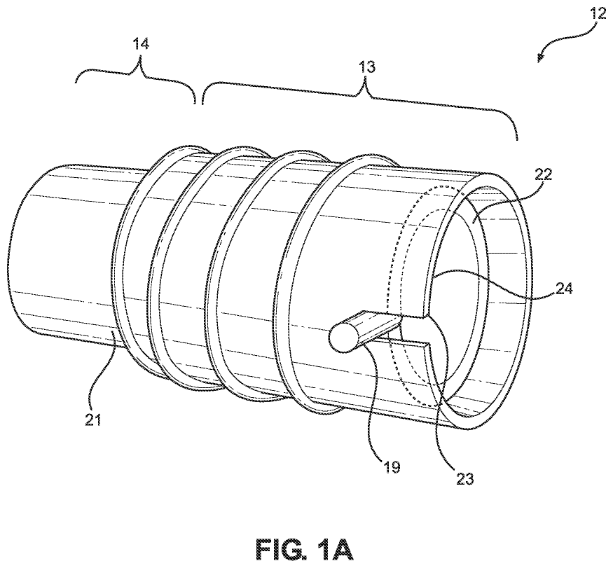

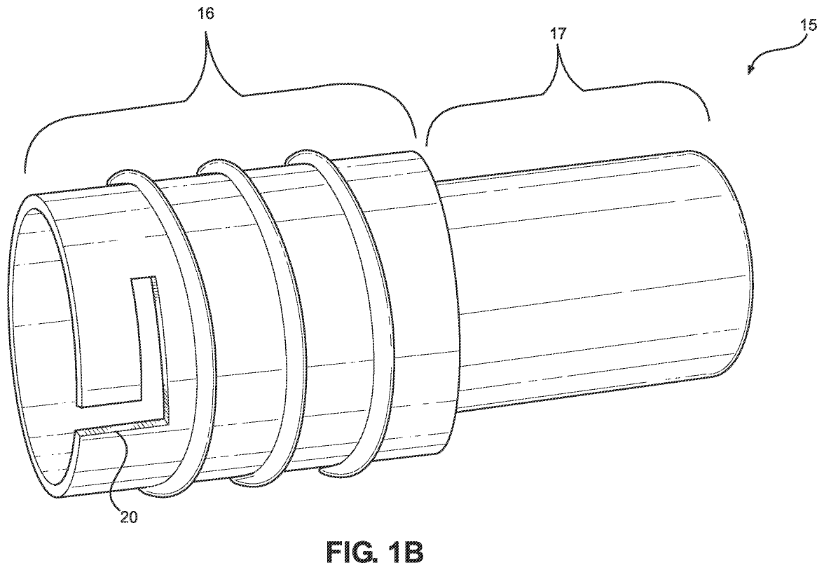

Referring now to FIGS. 1A and 11, there is shown a perspective view of the male connector body of an embodiment of the tubing connection adaptor system and a perspective view of the female connector body of an embodiment of the tubing connection adaptor system, respectively. The tubing connection adaptor system comprises a male connector body 12 removably securable to a female connector body 15. The male connector body 12 comprises a first end 13 opposite a second end 14. In the illustrated embodiment, each of the first and second ends 13, 14 comprise a tubular construction, wherein the first end 13 comprises a larger diameter than the second end 14. Particularly, in the shown embodiment, the second end 14 comprises a tubular member 21 extending into an interior volume of the first end 13, wherein the tubular member 21 defines a continuous channel extending through the male connector body 12 and the female connector body 15 when in a connected configuration, such that fluid is readily transported through the male and female connector bodies 12, 15. The female connector body 15 comprises a front end 16 opposite a rear end 17, wherein the front end 16 comprises a larger diameter than the rear end 17. Furthermore, the front end 16 is dimensioned to receive the first end 13 of the male connector body 12 therein when in the connected configuration, such that a watertight seal is formed therebetween. In this manner, the continuous channel defined between the male and female connector bodies 12, 15 when in the connected configuration minimizes leaks when the tubing connector adaptor system is in use. Furthermore, in the shown embodiment, the second end 14 and the rear end 17 comprise an identical diameter, such that the second and rear ends 14, 17 are securable within uniformly sized medical tubing.

A tab 19 extends from the first end 13 of the male connector body 12, wherein the tab 19 is dimensioned to frictionally engage a notch 20 disposed through the front end 16 of the female connector body 15. In this manner, the tab 19 locks within the notch 20, such that the male connector body 12 is securely affixed to the female connector body 15 throughout use. In the illustrated embodiment, the tab 19 is affixed to a distal end 22 of the tubular member 21 and extends through the first end 13. In this manner, a larger surface area of the tab 19 is affixed to the male connector body 12, such that the tab 19 is reinforced by the first end 13, thereby minimizing the risk of damage to the tab 19. In the illustrated embodiment, a cutout 23 is disposed through the first end 13 between a forward edge 24 of the first end 13 and the tab 19. The cutout 23 terminates at the tab 19 such that an entirety of the cutout 23 is disposed within the front end 16 of the female connector body 15 when in the connected configuration. In this manner, fluid leaks from the cutout 23 when the male and female connector bodies 12, 15 are in the connected configuration are prevented.

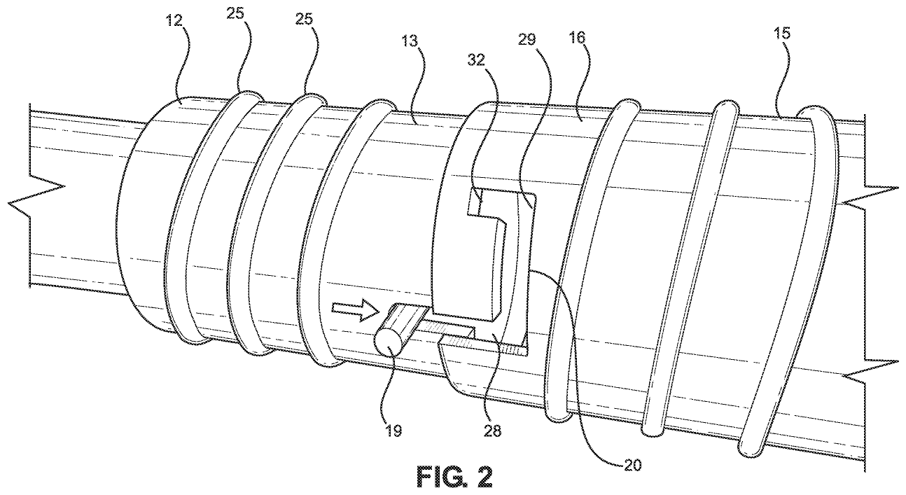

Referring now to FIG. 2, there is shown a perspective view of an embodiment of the tubing connection adaptor system in a partially connected configuration. As previously described, the tab 19 frictionally engages within the notch 20 when the male connector body 12 is affixed to the female connector body 15. In the illustrated embodiment, the notch 20 comprises a plurality of legs defining a J-shaped notch 20. In other embodiments, such as shown in FIG. 16, the notch 20 comprises a pair of legs defining an L-shape. In such embodiments, the tab 19 is maneuvered through the notch 20 to prevent the tab 19 from unintentionally disengaging during use. In the shown embodiment, the notch 20 comprises a first leg 28 extending from an edge of the front end 16 of the female connector body 15, wherein a second leg 29 extends perpendicularly from the first leg 28. A third leg 32 extends perpendicularly from a terminal end of the second leg 29 such that the first and third legs 28, 32 are disposed parallel to each other. In this manner, the third leg 32 prevents the tab 19 from shifting laterally along the notch 20 during use. In the shown embodiment, the third leg 32 comprises a length less than that of the first leg 28, such that the third leg 32 does not extend through an edge of the front end 16. A width of the notch 20 is dimensioned to a close tolerance of a diameter of the tab 19 such that the notch 20 frictionally engages the tab 19 therein. In some embodiments, a width of the notch 20 decreases towards a terminal end of the second or third legs 29, 32, such that the tab 19 is locked within the notch 20 during use. In other embodiments, a locking mechanism is spring-biased within the tab 19, wherein the locking mechanism comprises at least one extension selectively movable between an extended position and a retracted position, wherein the extension is spring-biased towards the extended position. As such, the locking mechanism frictionally engages the notch 20 when in the extended position. In such embodiments, a button is disposed on the first end 13 of the male connector body 12, wherein the button is operably connected to the locking mechanism, such that the extension selectively moves towards the retracted position when the button is actuated.

In the illustrated embodiment, a plurality of ridges 25 are disposed about an exterior of each of the first end 13 and the front end 16, wherein the plurality of ridges 25 provide additional surface area to increase frictional engagement with each of the male and female connector bodies 12, 15. In the shown embodiment, the plurality of ridges 25 comprise concentric rings about each of the front end 16 and the first end 13. The plurality of ridges 25 extend along the first end 13 rearward of the tab 19, such that the plurality of ridges 25 do not interfere with insertion of the first end 13 within the front end 16 of the female connector body 15. In the shown embodiment, an exterior surface of each of the plurality of ridges 25 comprises an arcuate edge to provide a comfortable gripping surface. Furthermore, in the illustrated embodiment, the plurality of ridges 25 are disposed at regular intervals along each of the first end 13 and the front end 16.

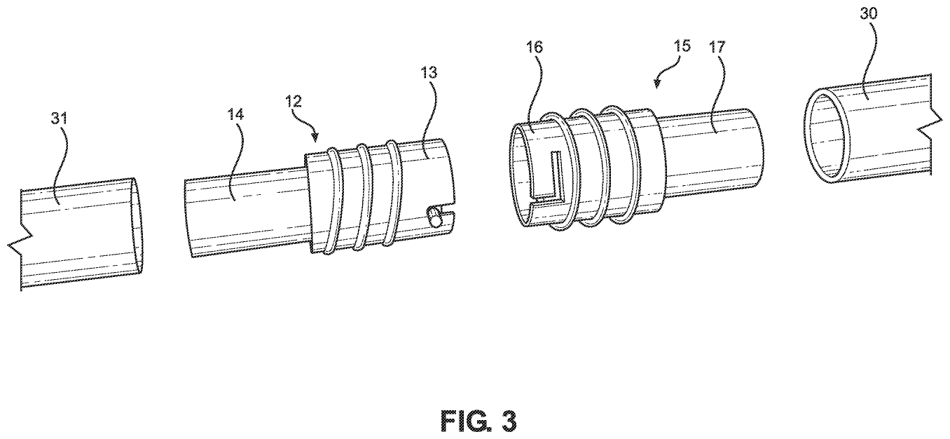

Referring now to FIG. 3, there is shown an exploded view of an embodiment of the tubing connection adaptor system in use. In one use, the male connector body 12 and the female connector body 15 are secured to medical tubing to secure the medical tubing together while providing a quick connect system. In the shown embodiment, the second end 14 of the male connector body 12 is secured within urine collection tubing 31 connected to a urine collection bag, while the rear end 17 of the female connector body 15 is secured within catheter tubing 30 secured to a patient. In the shown embodiment, each of the second end 14 and the rear end 17 are dimensioned within a close tolerance of an interior diameter of each of the urine collection tubing 31 and the catheter tubing 30, such that the second and rear ends 14, 17 are frictionally secured therein forming a watertight seal. Once secured within the desired medical tubing, the first end 13 of the male connector body 12 can be secured within the front end 16 of the female connector body 15 utilizing the tab and notch as previously described. The user can lock the male and female connector bodies 12, 15 together via rotating the tab through the notch, forming a watertight continuous channel extending through the male and female connector bodies 12, 15 that operably connects the urine collection tubing 31 to the catheter tubing 30. When the urine collection bag is at capacity, the user can disconnect the male connector body 12 from the female connector body 15 and secure a new urine collection bag to the catheter. As such, the effort required to change urine collection bags or other medical connections as well as the discomfort to the patient during such procedures is minimized.

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly, and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A tubing connection adaptor system, comprising:
a male connector body having a first end opposite a second end;
a female connector body having a front end opposite a rear end;
wherein the front end of the female connector body is dimensioned to operably engage the first end of the male connector body therein;
wherein a continuous channel is defined through the male connector body and the female connector body upon engagement therebetween;
a tab extending from the first end of the male connector body;
wherein the tab frictionally engages a notch disposed through the front end of the female connector body.

2. The tubing connection adaptor system of claim 1, wherein the first end comprises a greater diameter than the second end.

3. The tubing connection adaptor system of claim 2, wherein the second end defines a tubular member extending into an interior of the first end.

4. The tubing connection adaptor system of claim 3, wherein the tab extends from a distal end of the tubular member through the first end.

5. The tubing connection adaptor system of claim 4, further comprising a cutout extending from a forward edge of the first end and terminating at the tab.

6. The tubing connection adaptor system of claim 1, wherein the notch comprises a first leg and a second leg, wherein the first leg is disposed perpendicularly from the second leg.

7. The tubing connection adaptor system of claim 6, wherein the notch further comprises a third leg extending perpendicularly from a terminal end of the second leg.

8. The tubing connection adaptor system of claim 1, wherein the second end and the rear end comprise an identical diameter.

9. The tubing connection adaptor system of claim 1, wherein the front end comprises a diameter greater than the rear end.

10. A tubing connection adaptor system, comprising:
a male connector body having a first end opposite a second end;
a female connector body having a front end opposite a rear end;
wherein the front end of the female connector body is dimensioned to operably engage the first end of the male connector body therein;
wherein a continuous channel is defined through the male connector body and the female connector body upon engagement therebetween;
a tab extending from the first end of the male connector body;
wherein the tab frictionally engages a notch disposed through the front end of the female connector body;
a plurality of ridges disposed about an exterior surface of each of the first end and the front end.

11. The tubing connection adaptor system of claim 10, wherein the first end comprises a greater diameter than the second end.

12. The tubing connection adaptor system of claim 11, wherein the second end defines a tubular member extending into an interior of the first end.

13. The tubing connection adaptor system of claim 12, wherein the tab extends from a distal end of the tubular member through the first end.

14. The tubing connection adaptor system of claim 13, further comprising a cutout extending from a forward edge of the first end and terminating at the tab.

15. The tubing connection adaptor system of claim 10, wherein the plurality of ridges is concentrically disposed along each of the first end and the front end.

16. The tubing connection adaptor system of claim 10, wherein each ridge of the plurality of ridges defines an arcuate exterior.

17. The tubing connection adaptor system of claim 10, wherein the notch comprises a first leg and a second leg, wherein the first leg is disposed perpendicularly from the second leg.

18. The tubing connection adaptor system of claim 17, wherein the notch further comprises a third leg extending perpendicularly from a terminal end of the second leg.

19. The tubing connection adaptor system of claim 10, wherein the second end and the rear end comprise an identical diameter.

20. The tubing connection adaptor system of claim 10, wherein the front end comprises a diameter greater than the rear end.
